(19) United States
(12) Patent Application Publication
(10) Pub. No.: US 2012/0128606 A1
(43) Pub. Date: May 24, 2012

(54) Luna creme

(76) Inventors: Dayo Igiebor, Bronx, NY (US); Patrick Umoh, Bronx, NY (US)

(21) Appl. No.: 13/373,182

(22) Filed: Nov. 7, 2011

Related U.S. Application Data
(60) Provisional application No. 61/456,409, filed on Nov. 5, 2010.

(51) Int. Cl. A61K 8/97; A61Q 19/02; A61Q 19/08; A61Q 17/04

(52) U.S. Cl. 424/59; 424/62

(57) ABSTRACT

The invention provides a product line features Skin Beautifying Milk, Body Creme, Fade Creme and Maxi Tone. Ingredients include, but are not limited to: water, pentylene glycol, glyceryl stearate, mineral oil, PEG-100 stearate, glycerin, palmitic acid, sodium dihydroxycetyl phosphate, ehtylhexyl palmitate, stearic acid, petrolatum, xanthan gum, beeswax, cetearyl alcohol, phenoxyethanol, fragrance, dimethicone, methylparaben, tocopheryl acetate, tetrasodium EDTA, ehtylparaben, propylparaben, isobutylparaben, rumex crispus root extract, sodium hydroxide, and lactic acid.

### CLAIM OF PRIORITY

This patent application claims priority under 35 USC 119 (e) (1) from U.S. Provisional Patent Application Ser. No. 61/456,409 filed Nov. 5, 2010, of common inventorship herewith entitled, “Luna Creme.”

### FIELD OF THE INVENTION

The present invention pertains to the field of emollients, and more specifically to the field of emollients for use as face and body cremes.

### BACKGROUND OF THE INVENTION

The prior art has put forth several formulations for face and body cremes. Among these are:

U.S. Pat. No. 5,561,153 to Ruey J. Yu and Eugene J. VanScott describe a method of treating wrinkles using mucic acid or a topically effective salt thereof.

U.S. Pat. No. 5,670,541 to Ruey J. Yu and Eugene J. Van Scott describe a method of treating wrinkles using mucolactone.

US Patent application publication 2009/0301508 describes a cosmetic composition and preparation method and use thereof comprising a skin-whitening, freckly-removing cosmetic composition made from an effective amount of extract from Cistanche deserticola.

None of these prior art references describe the present invention.

### SUMMARY OF THE INVENTION

It is an object of the present invention to provide an improved skin crème for lightening liver spots and age spots, as well as darkened areas of the skin.

### BRIEF DESCRIPTION OF THE DRAWINGS

FIG. 1a is front perspective view of a bottle of skin beautifying milk in accordance with the present invention.

FIG. 1b is a front perspective view of a jar of body crème in accordance with the present invention.

FIG. 1c 3 is a front perspective view of a jar of fade crème in accordance with the present invention.

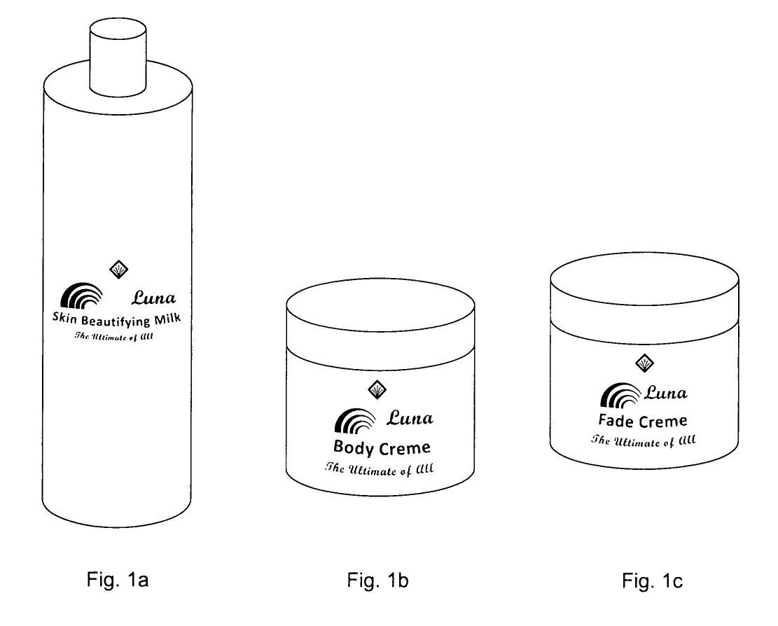

### DETAILED DESCRIPTION OF THE INVENTION

The Luna Creme product line features Skin Beautifying Milk, Body Creme, Fade Creme and Maxi Tone. This specially formulated topical moisturizer can be used on various parts of the body to reduce the appearance of darkened skin, liver and age spots. Reducing the appearance of fine lines and wrinkles, the Luna Creme is fast absorbing and non-greasy. The Luna Creme product line features the highest quality ingredients to moisturize and lighten skin, while keeping it fresh and youthful.

Ingredients include, but are not limited to: water, pentylene glycol, glyceryl stearate, mineral oil, PEG-100 stearate, glycerin, palmitic acid, sodium dihydroxycetyl phosphate, ehtylhexyl palmitate, stearic acid, petrolatum, xanthan gum, beeswax, cetearyl alcohol, phenoxyethanol, fragrance, dimethicone, methylparaben, tocopheryl acetate, tetrasodium EDTA, ehtylparaben, propylparaben, isobutylparaben, rumex crispus root extract, sodium hydroxide, and lactic acid.

The Luna Creme features an SPF rating of 15 or higher to protect skin, especially lightened skin, from the damaging rays of the sun. As an additional consideration, the Luna Creme line can be offered in a reduced-sized travel version, for convenient skin therapy while away from home. The Luna Creme line offers consumers a simple means of replenishing the skin with moisture, as well as provide them with a top of the line product which serves to lighten any dark unattractive areas of the skin. Used daily, this product beautifies, lightens and protects the user's skin. The Luna Creme line greatly reduces the signs of aging, including fine lines and wrinkles, age and liver spots, as well as darkened or discolored skin. Quickly absorbed by the skin, the Luna Creme is a top notch formula that leaves skin feeling hydrated, fresh and radiant.

Although this invention has been described with respect to specific embodiments, it is not intended to be limited thereto and various modifications which will become apparent to the person of ordinary skill in the art are intended to fall within the spirit and scope of the invention as described herein taken in conjunction with the accompanying drawings and the appended claims.

1. An improved crème for reducing wrinkles and lightening the skin, including water, pentylene glycol, glyceryl stearate, mineral oil, PEG-100 stearate, glycerin, palmitic acid, sodium dihydroxycetyl phosphate, ehtylhexyl palmitate, stearic acid, petrolatum, xanthan gum, beeswax, cetearyl alcohol, phenoxyethanol, fragrance, dimethicone, methylparaben, tocopheryl acetate, tetrasodium EDTA, ehtylparaben, propylparaben, isobutylparaben, rumex crispus root extract, sodium hydroxide, and lactic acid.

2. The crème of claim 1 further comprising sunscreen ingredients to render the creme an SPF rating of 15 or higher to protect skin, especially lightened skin, from the damaging rays of the sun.

3. The crème of claim 1 packaged in a reduced-sized travel version, for convenient skin therapy while away from home.
